(19) United States
(12) Patent Application Publication
(10) Pub. No.: US 2026/0317345 A1
(43) Pub. Date: Oct. 15, 2026

(54) N,S-DOPED ACENES, METHOD OF SYNTHESIS, ELECTROCHEMICAL SCREENING, AND USE OF SAME FOR BATTERY FABRICATION

(71) Applicant: Research Triangle Institute, Research Triangle Park, NC (US)

(72) Inventors: Paban SITAULA, Morrisville, NC (US); Mustapha SOUKRI, Cary, NC (US)

(21) Appl. No.: 19/484,030

(22) PCT Filed: May 13, 2024
(86) PCT No.: PCT/US2024/029026

§ 371 Date: Nov. 13, 2025

Related U.S. Application Data
(60) Provisional application No. 63/467,260, filed on May 17, 2023.

(51) Int. Cl. C07D 513/22; H01M 10/0525; H01M 10/0568

(52) U.S. Cl. CPC C07D 513/22; H01M 10/0525; H01M 10/0568; H01M 2300/0025

(57) ABSTRACT

Heteroatom-doped acenes, e.g., acenes doped with nitrogen and sulfur atoms, are described. The heteroatom-doped acenes can be redox-active and/or can include nitrogen substituents that can tune the solubility of the heteroatom-doped acene in non-protic polar solvents. Electrolytes and batteries comprising the heteroatom-doped acenes are also described. In addition, a method of synthesizing heteroatom-doped acenes via aromatic nucleophilic substitution reactions is described.

### CROSS-REFERENCE TO RELATED APPLICATION

This application claims the benefit of priority of U.S. provisional patent application No. 63/467,260 titled “N,S-DOPED ACENES, METHOD OF SYNTHESIS, ELECTROCHEMICAL SCREENING AND USE OF SAME FOR BATTERY FABRICATION” filed May 17, 2023, which is incorporated by reference herein.

### TECHNICAL FIELD

The presently disclosed subject matter relates to heteroatom-doped acenes, e.g., N,S-doped heptacene and other N,S-doped or N,S,O-doped acenes. The presently disclosed subject matter further relates to the synthesis of heteroatom-doped acenes and their use in energy storage and organo-optoelectronic devices.

### BACKGROUND

The development of clean energy storage systems is an increasingly desirable goal to reduce extensive dependence on natural resources, such as geothermal plants, fossil fuels, solar power, and wind power. Redox-flow battery (RFB) and lithium-ion battery (LIB) are rechargeable batteries with high-energy density and are majorly used in portable equipment. The market for these batteries is expected to witness significant growth owing to increase in use in smartphones, tablets/PCs, digital cameras, electric cars, and power tools. However, there are a limited number of redox-active molecular scaffolds for possible inclusion in such systems, and many of these have issues that impact their usefulness, e.g., related to solubility, electrochemical potential, and chemical and electrochemical stability in both neutral and charged forms.

Accordingly, there is an ongoing need for additional molecular scaffolds and particular compounds for use as electrolytes. In particular, there is a need for scaffolds and compounds with high oxidation potential and good solubility and stability.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a heteroatom-doped acene, wherein the heteroatom-doped acene comprises a fused ring moiety comprising at least seven fused rings, wherein said at least seven fused rings together comprise at least six heteroatoms selected from O, N, S, and B, and wherein the at least six heteroatoms comprise at least one N atom and at least one S atom.

In some embodiments, the fused ring moiety comprises a central phenothiazine moiety. In some embodiments, the heteroatom-doped acene is a heteroatom-doped heptacene or longer acene. In some embodiments, the heteroatom-doped acene is a heteroatom-doped heptacene or longer acene doped with at least one N atom and at least five S atoms.

In some embodiments, the presently disclosed subject matter provides a heteroatom-doped acene, wherein the heteroatom-doped acene has a structure of Formula (I):

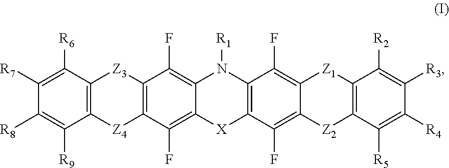

wherein: X is selected from N, S, and B; each of Z₁, Z₂, Z₃, and Z₄ is independently selected from O, N, and S; R₁ is selected from —(CH₂)ₚ—O—(CH₂)_{q}—R₁₀,

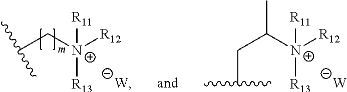

wherein p, q, and m are each an integer, independently selected from 1, 2, and 3; and R₁₀, R₁₁, R₁₂, and R₁₃ are independently selected from H, alkyl, and benzyl; R₂-R₉ are each independently selected from H, alkyl, alkoxy, alkylamino, alkylthio, aralkyl, aryl, aryloxy, arylamino, and arylthiol, or wherein R₇ and R₈ together with the carbon atoms to which they are attached and/or R₃ and R₄ together with the carbon atoms to which they are attached form a fused five- or six-membered aromatic or heteroaromatic ring, optionally selected from the group comprising benzene, substituted benzene, pyridine, substituted pyridine, thiophene, furan, and pyrrole; and W is an anionic moiety, optionally selected from the group comprising bis(trifluoromethane)sulfonimide, (TFSI), tetrafluoroborate (BF₄), Br, Cl, I, trifluorormethanesulfonate (OTf), toluenesulfonate (OTs), and methanesulfonate (OMs).

In some embodiments, at least one of X, Z₁, Z₂, Z₃, and Z₄ X is S. In some embodiments, X is S. In some embodiments, Z₁, Z₂, Z₃, and Z₄ are each S.

In some embodiments, X is S and each of Z₁, Z₂, Z₃, and Z₄ is S and the heteroatom-doped acene of Formula (I) has a structure of Formula (II):

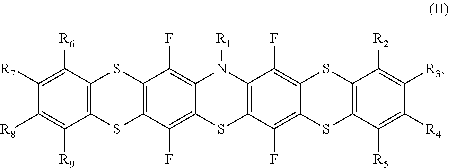

wherein R₁-R₉ are as defined for Formula (I). In some embodiments, R₁ is —(CH₂)—O—(CH₂)_{q}—O—R₁, wherein p and q are each 2. In some embodiments, R₁₀ is alkyl, optionally methyl. In some embodiments, R₂-R₉, are each independently selected from H, C1-C5 alkyl, and C1-C5 alkoxy. In some embodiments, R₂-R₉ are independently selected from H, methyl, ethyl, isopropyl, tert-butyl, methoxy, ethoxy, isopropyloxy, and tert-butoxy.

In some embodiments, R₇ and R₈ together with the carbon atoms to which they are attached and R₃ and R₄ together with the carbon atoms to which they are attached each form a fused five- or six-membered aromatic or heteroaromatic ring, optionally selected from the group comprising benzene, substituted benzene, pyridine, substituted pyridine, thiophene, furan, and pyrrole.

In some embodiments, the heteroatom-doped acene has a structure selected from:

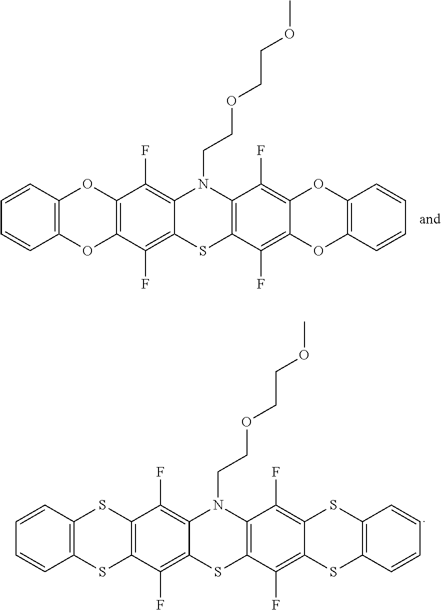 [SMILES: COCCOCCn1c2c(F)c3oc4ccccc4oc3c(F)c2sc2c(F)c3oc4ccccc4oc3c(F)c21 ; COCCOCCn1c2c(F)c3sc4ccccc4sc3c(F)c2sc2c(F)c3sc4ccccc4sc3c(F)c21]

In some embodiments, the heteroatom-doped acene can undergo a reversible redox reaction. In some embodiments, the heteroatom-doped acene is chemically stable in both charged and neutral forms. In some embodiments, the heteroatom-doped acene has an oxidation potential of about 0.7 V or more referenced to ferrocene (Fc)/ferrocenium (Fc+), optionally wherein the heteroatom-doped acene has an oxidation potential of about 0.7 V to about 1.75 V referenced to Fc/Fc+.

In some embodiments, the presently disclosed subject matter provides a radical cation of a heteroatom-doped acene of the presently disclosed subject matter.

In some embodiments, the presently disclosed subject matter provides an electrolyte comprising a heteroatom-doped acene of the presently disclosed subject matter. In some embodiments, the presently disclosed subject matter provides a battery comprising the electrolyte comprising a heteroatom-doped acene of the presently disclosed subject matter. In some embodiments, the battery is a lithium-ion battery (LIB) or an organic redox-flow battery (RFB).

In some embodiments, the presently disclosed subject matter provides a method of preparing a heteroatom-doped acene, wherein the method comprises: (a) contacting octafluorophenothiazine (OFPT) with a first reagent in the presence of a hydride base, wherein said first reagent has a structure selected from:

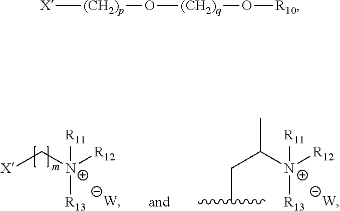

wherein X′ is Cl, Br, I, toluenesulfonate (OTs), or methanesulfonate (OMs); p, q, and m are each integers, independently selected from 1, 2, and 3; and R₁₀, R₁₁, R₁₂, and R₁₃ are each independently selected from H, alkyl, and benzyl; thereby providing a N-substituted OFPT; and (b) performing an aromatic nucleophilic substitution reaction by contacting the N-substituted OFPT with a substituted aromatic compound, wherein the substituted aromatic compound comprises a substituted benzene or fused aromatic compound comprising at least one fused benzene ring, and wherein said substituted aromatic compound further comprises at least two nucleophilic substituents selected from —OH, —NH₂, and —SH, wherein said at least two nucleophilic substituents are substituted on neighboring carbon atoms of the same benzene or fused benzene ring, thereby providing a heteroatom-doped acene, wherein said heteroatom-doped acene comprises a fused ring moiety comprising at least seven fused rings, wherein said at least seven fused rings together comprise at least six heteroatoms selected from O, N, S. and B, and wherein the at least six heteroatoms comprise at least one N atom and at least one S atom.

In some embodiments, the first reagent is methoxyethoxyethyl bromide (MEEBr). In some embodiments, the hydride base is sodium hydride (NaH). In some embodiments, the substituted aromatic compound is selected from benzene-1,2-dithiol and benzene-1,2-diol. In some embodiments, the heteroatom-doped acene is selected from:

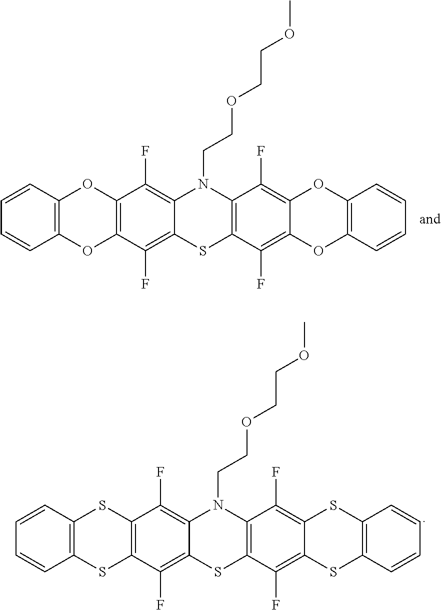 [SMILES: COCCOCCn1c2c(F)c3oc4ccccc4oc3c(F)c2sc2c(F)c3oc4ccccc4oc3c(F)c21 ; COCCOCCn1c2c(F)c3sc4ccccc4sc3c(F)c2sc2c(F)c3sc4ccccc4sc3c(F)c21]

Accordingly, it is an object of the presently disclosed subject matter to provide heteroatom-doped acenes doped at least with nitrogen and sulfur and/or having a structure of Formula (I), as well as to provide related radical cations, electrolytes, and batteries, and methods of preparing the heteroatom-doped acenes. Certain objects of the presently disclosed subject matter having been stated hereinabove, which are addressed in whole or in part by the presently disclosed subject matter, other objects and aspects will become evident as the description proceeds when taken in connection with the accompanying Examples as best described herein below.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the below drawings.

FIG. 1 is a graph showing the cyclic voltammograms (current (in microampere (μA)) versus potential (in volts (V)) versus ferrocene/ferrocenium (Cp₂Fe^{0/+}) in of 10 millimolar (mM) solution of an exemplary nitrogen, sulfur (N,S)-doped heteroheptacene of the presently disclosed subject matter (MEETFDTBPT) in 0.1 molar (M) TEABF₄ in acetonitrile at scan rates of 10 millivolts per second (mV/s) to 500 mV/s.

FIG. 2 is a graph showing the ultraviolet-visible (UV) spectra (absorbace (in arbitrary units (a.u.)) versus wavelength (in nanometers (nm))) of an exemplary nitrogen-sulfur (N,S)-doped heteroheptacene of the presently disclosed subject matter (i.e., MEETFDTBPT) and of its radical cation (MEETFDTBPT-2TFSI).

FIG. 3 is a graph showing the ultraviolet-visible (UV) spectra (absorbace (in arbitrary units (a.u.)) versus wavelength (in nanormeters (nm))) of a 10 millimolar (mM) solution of a radical cation of an exemplary nitrogen, sulfur (N,S)-doped heteroheptacene of the presently disclosed subject matter (MEETFDTBPT-2TFSI) in acetonitrile after 5 minutes (min), 15 min, 30 min, 1 hour (h), 2 h, 3 h, and 24 h.

### DETAILED DESCRIPTION

The presently disclosed subject matter will now be described more fully hereinafter with reference to the accompanying Figures and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist, unless as otherwise specifically indicated.

### I. Definitions

Following long-standing patent law convention, the terms “a”, “an”, and “the” refer to “one or more” when used in this application, including the claims. Thus, for example, reference to “a solvent” includes mixtures of one or more solvents, two or more solvents, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

The term “about”, as used herein when referring to a measurable value such as an amount of weight, molar equivalents, time, temperature, etc. is meant to encompass in one example variations of ±20% or ±10%, in another example ±5%, in another example ±1%, and in yet another example ±0.1% from the specified amount, as such variations are appropriate to perform the disclosed methods.

The term “and/or” when used to describe two or more activities, conditions, or outcomes refers to situations wherein both of the listed conditions are included or wherein only one of the two listed conditions are included.

The term “comprising”, which is synonymous with “including,” “containing,” or “characterized by” is inclusive or open-ended and does not exclude additional, unrecited elements or method steps. “Comprising” is a term of art used in claim language, which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” refers to C1-C20 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C1-C8 alkyl), e.g., 1, 2, 3, 4, 5, 6, 7, or 8 carbon atoms. In some embodiments, “lower alkyl” can refer to C1-C6 or C1-C5 alkyl groups. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C1-C8, C1-C6, or C1-C5 straight-chain or branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, nitro, cyano, amino, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, cyano, amino, alkylamino, dialkylamino, ester, acyl, amide, sulfonyl, sulfate, and mercapto.

“Cyclic” and “cycloalky!” refer to a non-aromatic mono- or multicyclic ring system of about 3 to about 10 carbon atoms, e.g., 3, 4, 5, 6, 7, 8, 9, or 10 carbon atoms. In some embodiments, the cycloalkyl ring system comprises between 3 and 6 carbon atoms. The cycloalkyl group can be optionally partially unsaturated. The cycloalkyl group also can be optionally substituted with an alkyl group substituent as defined herein. There can be optionally inserted along the cyclic alkyl chain one or more oxygen, sulfur, or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinbelow, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds (i.e., “heteroaryl”). The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyridine, imidazole, benzimidazole, isothiazole, isoxazole, pyrazole, pyrazine, triazine, thiazole, pyrimidine, quinoline, isoquinoline, indole, carbazole, napthyl, and the like.

“Heterocyclic”, “heterocycle”, or “heterocyclo” as used herein alone or as part of another group, refers to an aliphatic (e.g., fully or partially saturated heterocyclo) or aromatic (e.g., heteroaryl) monocyclic- or a bicyclic-ring system comprising one or more heteroatoms (e.g., 1, 2, or 3 heteroatoms selected from oxygen, sulfur, and substituted or unsubstituted nitrogen) inserted along the cyclic alkyl or aryl carbon chain. Monocyclic ring systems are exemplified by any 5- or 6-membered ring containing 1, 2, 3, or 4 heteroatoms independently selected from oxygen, nitrogen, and sulfur. The 5 membered ring has from 0-2 double bonds and the 6 membered ring has from 0-3 double bonds. Representative examples of monocyclic ring systems include, but are not limited to, ethylene oxide, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydropyran, tetrahydrothiophene (also known as thiolane), tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, carbazole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and can be optionally substituted with one or more alkyl and/or aryl group substituents.

“Substituted heterocyclic” as used herein refers to a heterocyclic group wherein one or more hydrogen atoms are replaced by an alkyl or aryl group substituent.

The term “heteroaryl” refers to an aromatic monocyclic- or a bicyclic-ring system (a fused, bridged or spirocyclic ring system) comprising one or more heteroatoms (e.g., 1, 2, or 3 heteroatoms selected from oxygen, sulfur, and substituted or unsubstituted nitrogen, wherein N-oxides, sulfur oxides and dioxides are permissible heteroatom substitutions) inserted along the cyclic aryl carbon chain. In some embodiments, the monocyclic heteroaryl group is a five to seven-membered aromatic ring. Representative heteroaryl groups include but are not limited to furan, thiophene, pyrrole, imidazole, pyrazole, triazole, tetrazole, oxazole, isoxazole, oxadiazole, thiaciazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyrindine, and pyrazolopyrimidine.

The term “substituted heteroaryl” refers to a heteroaryl group as defined herein wherein one or more hydrogen atoms is replaced by an aryl group substituent.

“Aralkyl” refers to an aryl-alkyl- or an -alkyl-aryl group wherein aryl and alkyl are as previously described and can include substituted aryl and substituted alkyl. Thus, “substituted aralkyl” can refer to an aralkyl group comprising one or more alkyl or aryl group substituents. Exemplary aralkyl groups include benzyl, phenylethyl, and naphthylmethyl.

“Alkylene” can refer to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated (i.e., include alkene or alkyne groups) and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Arylene” refers to a bivalent aryl group, which can be substituted or unsubstituted.

The term “aralkylene” refers to a bivalent group that comprises a combination of alkylene and arylene groups (e.g., -arylene-alkylene-, alkylene-arylene-alkylene-, arylene-alkylene-arylene-, etc.).

Similarly, the terms “cycloalkylene”, “heterocycloalkylene” and “heteroarylene” refer to bivalent cycloalkyl, heterocyclic, and heteroaryl groups, which can optionally be substituted with one or more alkyl or aryl group substituents.

As used herein, the term “acyl” refers to an organic carboxylic acid group wherein the —OH of the carboxylic acid group has been replaced with another substituent. Thus, an acyl group can be represented by RC(═O)—, wherein R is an alkyl, substituted alkyl, aralkyl, substituted aralkyl, aryl or substituted aryl group as defined herein. As such, the term “acyl” specifically includes arylacyl groups, such as a phenacyl group. Specific examples of acyl groups include acetyl (i.e., —C(═O)CH₃) and benzoyl.

“Alkoxyl” refers to an alkyl-O— group wherein alkyl is as previously described, including substituted alkyl. The term “alkoxyl” as used herein can refer to, for example, methoxyl, ethoxyl, propoxyl, isopropoxyl, butoxyl, t-butoxyl, and pentoxyl. The terms “oxyalkyl” and “alkoxy” can be used interchangeably with “alkoxyl”.

“Aryloxyl” and “aryloxy” refer to an aryl-O— group wherein the aryl group is as previously described, including a substituted aryl. The term “aryloxyl” as used herein can refer to phenyloxyl or hexyloxyl, and to alkyl, substituted alkyl, or alkoxyl substituted phenyloxyl or hexyloxyl.

“Aralkyloxyl” or “aralkoxy” refer to an aralkyl-O— group wherein the aralkyl group is as previously described. An exemplary aralkyloxyl group is benzyloxyl.

The term “carbonyl” refers to the group —C(═O)—. The term “carbonyl carbon” refers to a carbon atom of a carbonyl group. Other groups such as, but not limited to, acyl groups, anhydrides, aldehydes, esters, lactones, amides, ketones, carbonates, and carboxylic acids, include a carbonyl group.

The terms “carboxyl” and “carboxylic acid” refer to the —C(═O)OH or —C(═O)O— group. In some embodiments, the term “carboxylate” refers to the C(═O)O— group.

The terms “halo” or “halogen” as used herein refer to fluoro, chloro, bromo, and iodo groups.

The term “amine” refers to a molecule having the formula N(R)₃, or a protonated form thereof, or a group having the formula —N(R)₂, wherein each R is independently H, alkyl, substituted alkyl, aryl, substituted aryl, aralkyl, substituted aralkyl, or wherein two R groups together form an alkylene or arylene group. The term “primary amine” refers to an amine wherein at least two R groups are H. The term “secondary amine” refers to an amine wherein only one R group is H. The terms “hydroxyl” and “hydroxy” refer to the —OH group.

The terms “mercapto” and “thiol” refer to the —SH group.

The term “oxo” refers to a compound described previously herein wherein a carbon atom is replaced by an oxygen atom.

The terms “bonding” or “bonded” and variations thereof can refer to either covalent or non-covalent bonding. In some cases, the term “bonding” refers to bonding via a coordinate bond. The term “conjugation” can refer to a bonding process, as well, such as the formation of a covalent linkage or a coordinate bond.

### II. Heteroatom-Doped Acenes, Related Compositions, and Devices

Nanographene or polycyclic aromatic hydrocarbons (PAHs) are organic molecules bearing more than two aromatic rings fused together. In particular, the term “acene” refers to a class of PAHs bearing fused benzene rings arranged in a straight line (i.e., rectilinearly), i.e., compounds having a core structure:

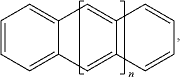

wherein n is an integer greater than 1. The acene structure can result in a unique butterfly topology. Acenes contain only one set of Clar's sextets, resulting in properties such as a high highest occupied molecular orbital (HOMO)—lowest unoccupied molecular orbital (LUMO) energy gap and an ability to behave as an open-shell structure. Due to the open-shell nature of these molecules, the longer the acene grows, the greater its stability.

Recently, the approach of introducing heteroatoms, such as boron (B), nitrogen (N), oxygen (O), and sulfur (S), into acene scaffolds to replace benzene ring carbon atoms has been explored, e.g., to provide new materials for opto-electronic applications (e.g., light-emitting diodes (LEDs). Heteroatom-doped acenes (also referred to herein as “heteroacenes”) and other PAHs can also have reversible redox activity, which can render them useful in energy storage applications, such as in electrolytes for batteries, e.g., for rechargeable batteries, including redox-flow batteries and lithium-ion batteries.

In some embodiments, the presently disclosed subject matter provides heteroatom-doped acenes (e.g., N, S- or N, S, O-doped heteroacenes) that comprise multiple redox-active subunits, which can have multi-electron redox activity. Due to their redox activity, in some embodiments, the heteroatom-doped acenes described herein can also be referred to as “redoxamers.” For instance, in some embodiments, two different redox-active subunits or substructures, i.e., thianthrene and phenothiazine, are combined in a single heteroacene. In some embodiments, a plurality of at least one type of redox-active subunit are present in the full heteroacene structure. In some embodiments, the heteroacene can include one or more dibenzo-1,4-dioxin (also known as oxanthrene) subunit or one or more thianthrene subunit. Structures of exemplary redox active subunits, i.e., thianthrene, phenothiazine, dibenzo-1,4-dioxin, and phenazine, are shown below in Scheme 1. In some embodiments, two adjoining subunits can share a single non-doped benzene ring. In some embodiments, one or more inductively electron withdrawing group (e.g., halo) is introduced as an acene substituent to boost the oxidation potential.

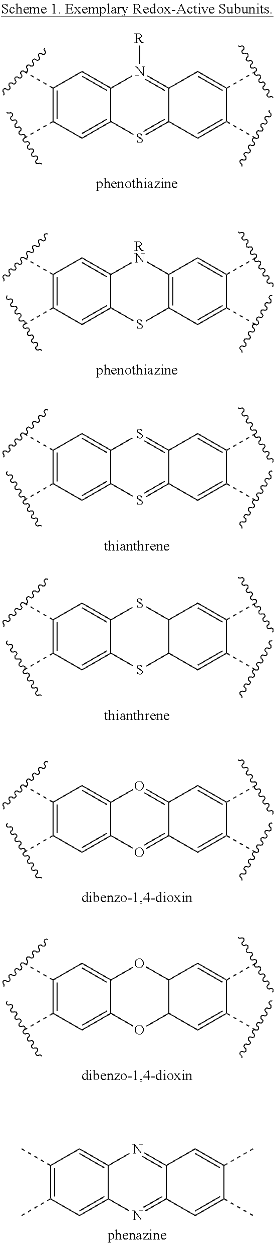

Thus, in some embodiments, the presently disclosed subject matter provides a N,S-doped heptacene or longer acene (e.g., a N,S-doped octacene, a N,S-doped nonacene, a N,S-doped decacene, or an N,S-doped dodecacene). In some embodiments, the N,S-doped acene further comprises one or more oxygen atoms (i.e., the N,S-doped acene is a N,S,O-doped acene) and/or more than one sulfur atom. In some embodiments, the N,S-doped heteroacene comprises a central phenothiazine subunit. In some embodiments, the doped heteroacene comprises a substituent on a nitrogen atom in the acene scaffold (e.g., in a central phenothiazine subunit) that can be used to tailor the solubility of the acene in a solvent of interest, e.g., in polar aprotic solvents, such as those used in lithium-ion battery (LIB) or organic redox-flow battery (RFB) applications.

Accordingly, in some embodiments, the presently disclosed subject matter provides a heteroatom-doped acene (i.e., a compound comprising a heteroatom-doped acene scaffold or moiety) doped with at least one nitrogen atom and at least one sulfur atom (i.e., a N,S-doped acene). For instance, in some embodiments, the heteroatom-doped acene comprises a fused ring moiety comprising at least seven fused rings, e.g., at least seven fused six-membered aromatic rings arranged in a straight line. In some embodiments, the heteroatom-doped acene comprises a heptacene or longer acene doped with at least one nitrogen atom and at least one sulfur atom. Thus, the heteroatom-doped acene can be based on a parent non-doped acene selected from heptacene (i.e., an acene comprising seven linearly fused benzene rings) or a longer non-doped acene (e.g., octacene, nonacene, decacene, dodecacene, etc.), wherein at least one carbon atom that is part of a fused ring in the parent non-doped acene is replaced by a nitrogen atom and at least one carbon atom that is part of a fused ring in the non-doped acene is replaced by a sulfur atom. The heteroatom-doped acene can also include one or more additional heteroatoms, such as one or more additional nitrogen atoms, one or more additional sulfur atoms, one or more oxygen atoms, or one or more boron atoms.

In some embodiments, the heteroatom-doped acene comprises one ring moiety comprising at least two heteroatoms (i.e., N and S). In some embodiments, more than one of the fused rings in the heteroatom-doped acene comprises one or more heteroatoms. In some embodiments, at least three of the fused rings in the heteroatom-doped acene comprise one or more heteroatoms. In some embodiments, two or more of the heteroatom-doped fused rings comprise two heteroatoms. In some embodiments, three of the heteroatom-doped fused rings comprise two heteroatoms.

In some embodiments, the presently disclosed subject matter provides a heteroatom-doped acene, wherein the heteroatom-doped acene comprises a fused ring moiety comprising at least seven fused rings (e.g., at least seven fused aromatic rings), wherein said at least seven fused rings together comprise least six heteroatoms selected from the group comprising or consisting of O, N, S, and B, wherein the at least six heteroatoms comprise at least one N atom and at least one S atom. In some embodiments, the fused ring moiety comprises a central phenothiazine moiety. For example, in some embodiments, the heteroatom-doped acene comprises an odd number of fused rings (e.g., fused aromatic rings) and the three fused rings in the center of the fused ring moiety form a phenothiazine group.

In some embodiments, the heteroatom-doped acene is an acene comprising more than seven fused rings (i.e., a heteroatom-doped acene comprising eight linearly fused rings or more). In some embodiments, the heteroatom-doped acene is doped with one N atom and at least five S atoms.

In some embodiments, the heteroatom-doped acene is doped with one N atoms, one S atom, and at least four O atoms.

In some embodiments, the presently disclosed subject matter provides a heteroatom-doped acene having a structure of Formula (I):

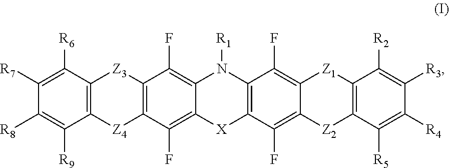

wherein: X is selected from N, S, and B; each of Z₁, Z₂, Z₃, and Z₄ is independently selected from O, N, and S; R₁ is selected from —(CH₂)ₚ—O—(CH₂)_{q}—O—R₁₀,

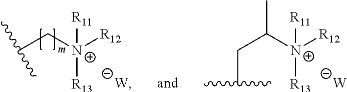

wherein p, q, and m are each an integer, independently selected from 1, 2, and 3; and R₁₀, R₁₁, R₁₂, and R₁₃ are independently selected from the group comprising, but not limited to, H, alkyl, and benzyl; R₂-R₉ are each independently selected from the group comprising, but not limited to, H, alkyl, alkoxy, alkylamino, alkylthio, aralkyl, aryl, aryloxy, arylamino, and arylthiol, or wherein R₇ and R₈ together with the carbon atoms to which they are attached and/or R₃ and R₄ together with the carbon atoms to which they are attached form a fused five- or six-membered aromatic or heteroaromatic ring; and W is an anionic moiety. In some embodiments, W is an anionic moiety selected from the group comprising bis(trifluorornethane)sulfonimide, (TFSI), tetrafluoroborate (BF₄), Br, Cl, I, trifluoromethanesulfonate (OTf), toluenesulfonate (OTs), and methanesulfonate (OMs). When R₇ and R₈ together with the carbon atoms to which they are attached and/or R₃ and R₄ together with the carbon atoms to which they are attached form a fused five- or six-membered ring, the fused ring can be unsubstituted or substituted. In some embodiments, the fused five- or six-membered ring is selected from benzene, substituted benzene, pyridine, substituted pyridine, thiophene, furan, and pyrrole.

In some embodiments, the heteroatom-doped acene of Formula (I) comprises at least one S. Thus, in some embodiments, at least one of X, Z₁, Z₂, Z₃, and Z₄ is S. In some embodiments, X is S (i.e., so that the structure of Formula (I) comprises a central phenothiazine). In some embodiments, one or more of Z₁, Z₂, Z₃, and Z₄ is S. In some embodiments, two of Z₁, Z₂, Z₃, and Z₄ are each S. In some embodiments, Z₁, Z₂, Z₃, and Z₄ are each S. In some embodiments, X is S and each of Z₁, Z₂, Z₃, and Z₄ is S and the compound of Formula (I) has a structure of Formula (11):

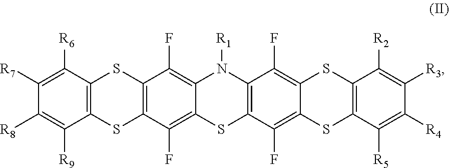

wherein R₁-R₉ are as defined above for Formula (I). Thus, in some embodiments, the compound of Formula (I) has a structure of Formula (II) wherein R₁ is selected from —(CH₂)ₚ—O—(CH₂)_{q}—O—R₁₀,

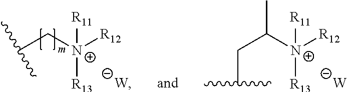

wherein p, q, and m are each an integer, independently selected from 1, 2, and 3; and R₁₀, R₁₁, R₁₂, and R₁₃ are independently selected from the group comprising, but not limited to, H, alkyl, and benzyl; R₂-R₉ are each independently selected from the group comprising, but not limited to, H, alkyl, alkoxy, alkylamino, alkylthio, aralkyl, aryl, aryloxy, arylamino, and arylthiol, or wherein R₇ and R₈ together with the carbon atoms to which they are attached and/or R₃ and R₄ together with the carbon atoms to which they are attached form a fused five- or six-membered aromatic or heteroaromatic ring; and W is an anionic moiety. In some embodiments, W is an anionic moiety selected from the group comprising TFSI, BF₄, Br, Cl, I, OTf, OTs, and OMs.

In some embodiments, one or more or two or more of Z₁, Z₂, Z₃, and Z₄ is O. In some embodiments, each of Z₁, Z₂, Z₃, and Z₄ is O. In some embodiments, X is S and each of Z₁, Z₂, Z₃, and Z₄ is O and the heteroatom-doped acene of Formula (I) has a structure of Formula (III):

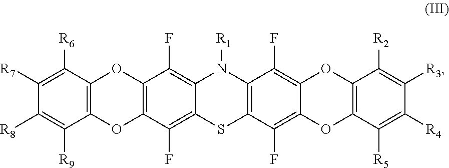

wherein R₁-R₉ are as defined above for Formula (I). Thus, in some embodiments, the compound of Formula (I) has a structure of Formula (III) wherein R₁ is selected from —(CH₂)ₚ—O—(CH₂)_{q}—O—R₁₀,

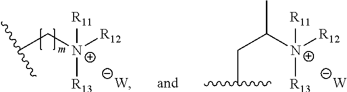

wherein p, q, and m are each an integer, independently selected from 1, 2, and 3; and R₁₀, R₁₁, R₁₂, and R₁₃ are independently selected from the group comprising, but not limited to, H, alkyl, and benzyl; R₂-R₉ are each independently selected from the group comprising, but not limited to, H, alkyl, alkoxy, alkylamino, alkylthio, aralkyl, aryl, aryloxy, arylamino, and arylthiol, or wherein R₇ and R₈ together with the carbon atoms to which they are attached and/or R₃ and R₄ together with the carbon atoms to which they are attached form a fused five- or six-membered aromatic or heteroaromatic ring; and W is an anionic moiety. In some embodiments, W is an anionic moiety selected from the group comprising TFSI, BF₄, Br, Cl, I, OTf, OTs, and OMs.

In some embodiments, the substituent of the nitrogen atom in the central ring of the structure of Formula (I)-(III), i.e., R₁, can be selected to tailor the solubility of the heteroatom-doped acene, e.g., to improve the solubility of the heteroatom-doped acene in aprotic polar solvents, such as, but not limited to, acetonitrile, dimethylformamide, dimethylsulfoxide, tetrahydrofuran, ethyl acetate, and carbonate solvents, such as, dimethyl carbonate, ethyl methyl carbonate, propylene carbonate, fluoroethylene carbonate, etc. In some embodiments, R₁ is a moiety based on ethylene glycol and has a structure R₁ is —(CH₂)ₚ—O—(CH₂)_{q}—O—R₁₀, wherein p and q are each 2. In some embodiments, R₁₀ is alkyl, e.g., a branched or straight chain C1-C5 alkyl, e.g, methyl, ethyl, propyl (e.g., isopropyl), butyl (e.g., tert-butyl), or pentyl. In some embodiments, R₁₀ is methyl. Thus, in some embodiments, R₁ is 2-(2-methoxyethoxyl)ethyl (i.e., —(CH₂)₂—O—(CH₂)₂—OMe).

In some embodiments, R₂-R₉ are each independently selected from H, C1-C5 alkyl, and C1-C5 alkoxy. Thus, for example, in some embodiments, R₂-R₉ are each independently selected from H, methyl, ethyl, n-propyl, isopropyl, n-butyl, sec-butyl, isobutyl, tert-butyl, n-pentyl, neopentyl, sec-pentyl, tert-pentyl, 3-pentyl, sec-isopentyl, methoxy, ethoxy, n-propyloxy, isopropyloxy, n-butyloxy, sec-butyloxy, isobutyloxy, tert-butyloxy, n-pentyloxy, neopentyloxy, sec-pentyloxy, tert-pentyloxy, 3-pentyloxy, and sec-isopentyloxy. In some embodiments, R₂-R₉ are each independently selected from H, methyl, ethyl, isopropyl, tert-butyl, methoxy, ethoxy, isopropyloxy, and tert-butoxy.

In some embodiments, (while the remaining R groups of R₂-R₉, e.g., R₂, R₅, R₆, and R₉, are still independently selected from H, C1-C5 alkyl, and C1-C5 alkoxy), R₇ and R₈ together with the carbon atoms to which they are attached and/or R₃ and R₄ together with the carbon atoms to which they are attached each form a fused five- or six-membered aromatic or heteroaromatic ring. In some embodiments, R₇ and R₈ together with the carbon atoms to which they are attached and R₃ and R₄ together with the carbon atoms to which they are attached each form a fused five- or six-membered aromatic or heteroaromatic ring. In some embodiments, the fused five- or six-membered aromatic or heteroaromatic ring is selected from the group comprising benzene, pyridine, pyridine, thiophene, furan, and pyrrole and can optionally be substituted with one or more aryl group substituents, e.g., alkyl, alkoxy, alkylamino, alkylthio, aralkyl, aryl, aryloxy, arylamino, and arylthiol. In some embodiments, the fused five- or six-membered aromatic or heteroaromatic ring is selected from benzene, substituted benzene, pyridine, substituted pyridine, thiophene, furan, and pyrrole. Thus, for example, the compound of Formula (I)-(III) can include 9 fused rings arranged linearly. For instance, the compound having a structure of Formula (I) can have a structure of Formula (Ia):

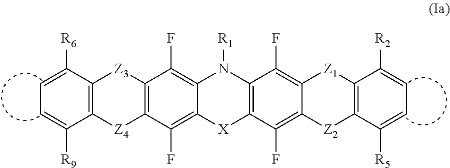

wherein:

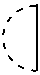

is selected from the group comprising substituted or unsubstituted benzene, pyridine, furan, thiophene, and pyrrole, X is selected from N, S, and B; each of Z₁, Z₂, Z₃, and Z₄ is independently selected from O, N, and S; R₁ is selected from —(CH)ₚ—O—(CH₂)_{q}—O—R₁₀,

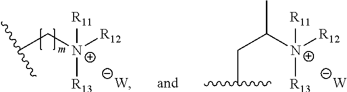

wherein p, q, and m are each an integer, independently selected from 1, 2, and 3; R₁₀, R₁₁, R₁₂, and R₁₃ are independently selected from the group comprising, but not limited to, H, alkyl, and benzyl; R₂, R₅, R₆, and R₉ are each independently selected from the group comprising, but not limited to, H, alkyl, alkoxy, alkylamino, alkylthio, aralkyl, aryl, aryloxy, arylamino, and arylthiol; and W is an anionic moiety. In some embodiments, W is an anionic moiety selected from the group comprising TFSI, BF₄, Br, Cl, O, OTf, OTs, and OMs.

In some embodiments,

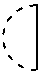

is selected from benzene, substituted benzene, pyridine, substituted pyridine, furan, thiophene, and pyrrole.

In some embodiments, the compound of Formula (Ia) has a structure of one of the following Formulas (Ia-1), (Ia-2), and (Ia-3):

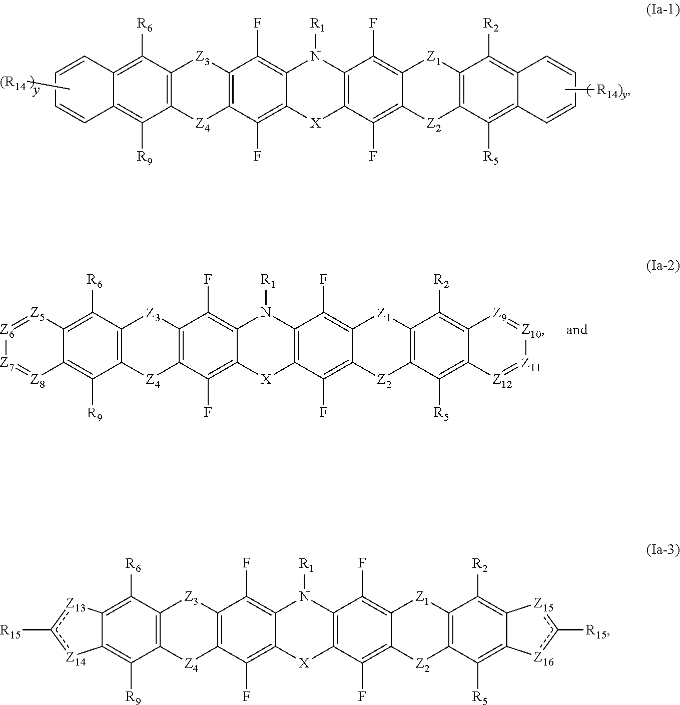

wherein R₁, R₂, R₅, R₆, R₉, X, and Z₁-Z₄ are as defined for Formula (O), and wherein [symbol] is a bond that can be present or absent; y is 0, 1, 2, 3, or 4; each R₁₄ and R₁₅ is selected from the group comprising alkyl, alkoxy, alkylamino, alkylthio, aralkyl, aryl, aryloxy, arylamino, and arylthiol; each of Z₅-Z₁₂ is selected from —C(R₁₆)— and —N(R₁₆)—, wherein each R₁₆ is selected from H, alkyl, alkoxy, alkayamino, alkylthio, aralkyl, aryl, aryloxy, arylamino, and arylthiol, subject to the proviso that at least one of Z₅-Z₈ and one of Z₉-Z₁₂ is —N(R₁₆)—; and Z₁₃, Z₁₄, Z₁₅, and Z₁₆ are each selected from —C(R₁₆)—, —N(R₁₆)—, —O—, and —S—; subject to the proviso that one of Z₁₃ and Z₁₄ and one of Z₁₅ and Z₁₆, is —C(R₁₆)— and one of Z₁₃ and Z₁₄ and one of Z₁₅ and Z₁₆ is —N(R₁₆)—, —O—, and —S—. Thus, the compounds of Formula (Ia-3) can have structures, such as, but not limited to:

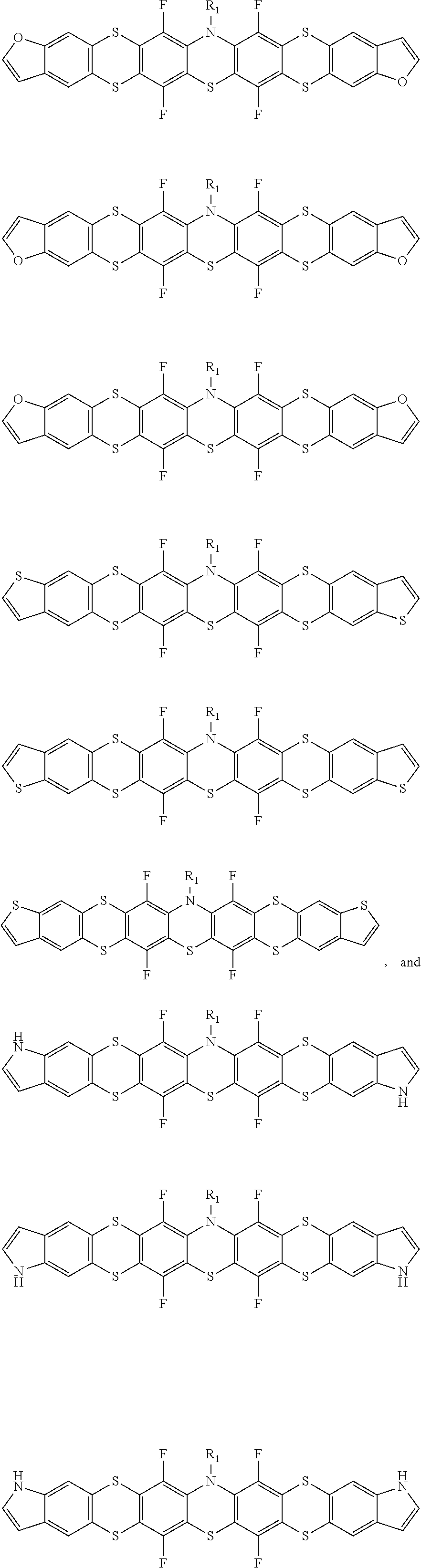

In some embodiments, each of R₂-R₉ is H. In some embodiments, the heteroatom-doped acene has a structure selected from:

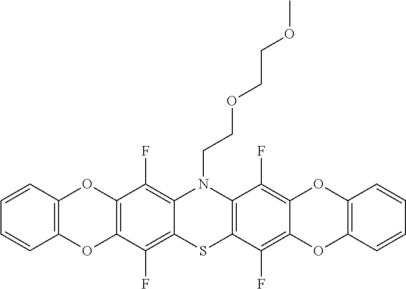 [SMILES: COCCOCCn1c2c(F)c3oc4ccccc4oc3c(F)c2sc2c(F)c3oc4ccccc4oc3c(F)c21]

(also referred to herein as MEETFDOBPT, i.e., 6,8,15,17-tetrafluoro-16-(2-(2-methoxyethoxy)ethyl)-16H-benzo[5,6][1,4]dioxino[2,3-b]benzo[5,6][1,4]dioxino-[2,3-i]phenothiazine) and

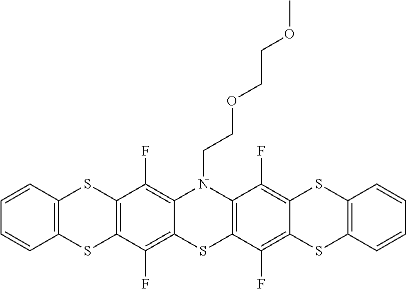 [SMILES: COCCOCCn1c2c(F)c3sc4ccccc4sc3c(F)c2sc2c(F)c3sc4ccccc4sc3c(F)c21]

(also referred to herein as MEETFDTBPT, i.e., 6,8,15,17-tetrafluoro-16-(2-(2-methoxyethoxy)ethyl)-16H-benzo[5,6][1,4]dithiino[2,3-b]benzo[5,6][1,4]dithiino-[2,3-i]phenothiazine).

In some embodiments, the heteroatom-doped acene of the presently disclosed subject matter (e.g., of Formulas (I)-(III) is redox active, e.g., can undergo a reversible redox reaction. In some embodiments, the heteroatom-doped acene is chemically stable in both charged and neutral forms. Thus, for example, the heteroatom-doped acene does not react or decompose when stored at room temperature or below for a prolonged period of time (e.g., at least 24 hours, at least one week, at least one month, or more) or dissolved in an organic solvent (e.g., acetonitrile) at room temperature or below for a prolonged period (e.g., at least 24 hours or more). In some embodiments, the chemical stability can be determined by the lack of change in a UV-Vis spectrum of the compound or a solution thereof over time (e.g., over one or more hours or one or more days).

In some embodiments, the heteroatom-doped acene has an oxidation potential of about 0.7 V or more referenced to ferrocene (Fc)/ferrocenium (Fc+) (where Fc/Fc+ can also be referred to as Cp₂Fe^{0/+}). In some embodiments, the heteroatom-doped acene has an oxidation potential of about 0.7 V to about 1.75 V (e.g., about 0.7 V, about 0.8 V, about 0.9 V, about 1.0 V, about 1.1 V, about 1.2 V, about 1.3V, about 1.4 V, about 1.5 V, about 1.6 V, about 1.7 V, or about 1.75 V) referenced to Fc/Fc+. In some embodiments, the heteroatom-doped acene has an oxidation potential of about 1.61 V referenced to Fc/Fc+.

In some embodiments, the presently disclosed subject matter provides a radical cation (e.g., a chemically stable radical cation) of a heteroatom-doped acene as described hereinabove (e.g., having a structure of Formula (I)). The radical cation can be formed, for example, by contacting the heteroatom-doped acene with a chemical oxidant (e.g., silver bistriflimide, tris-(2,4-dibromophenyl)aminium hexachloroantimonate (also known as Magic Green), antimony pentafluoride, nitrosonium tetrafluoroborate, etc).

In some embodiments, the presently disclosed subject matter provides an electrolyte comprising a heteroatom-doped acene as described herein (i.e., a redox active heteroatom-doped acene, such as, but not limited to MEETFDTBPT). In some embodiments, the electrolyte further comprises a polar aprotic solvent such as a carbonate (e.g., dimethyl carbonate, propylene carbonate, ethyl methyl carbonate, etc.) acetonitrile, a mixture of dichloromethane and acetonitrile, and the like.

In some embodiments, the presently disclosed subject matter provides an energy storage device comprising the heteroatom-doped acene or the electrolyte comprising said heteroatom-doped acene. In some embodiments, the energy storage device is a battery, e.g., a LIB or RFB. In some embodiments, the presently disclosed subject matter provides an organic-optoelectronic device comprising a heteroatom-doped acene as described herein.

### III. Methods of Preparing Heteroatom-Doped Acenes

According to one aspect of the presently disclosed subject matter, regioselective, mild, and effective syntheses of heteroacenes (e.g., N,S-doped heteroheptacenes and N,S,O-doped heteroheptacenes) are provided. The syntheses involve aromatic nucleophilic substitution (SNAr) reactions to extend the acene scaffold structure.

In some embodiments, the synthesis proceeds from a phenothiazine, although another N-doped triacene can also be used. See Scheme 2, below. For example, the synthesis of a N,S-doped heptacene can be carried out by first preparing octafluorophenothiazine (OFPT), which can be synthesized as previously described. See Kaur et al., 2016. Then, the amine functional group in OFPT can be derivatized by reacting OFPT with a reagent R′X, i.e., an alkyl halide or other compound that comprises a good leaving group (e.g., a mesylate or tosylate), in the presence of a strong base, e.g., a hydride base, such as sodium hydride (NaH) or potassium hydride (KH) to provide a N-substituted OFPT. For example, in some embodiments, the reagent is an alkyl halide (e.g., an alkyl bromide) further comprising additional functional groups (e.g., one or more ether or amine groups) that can form a desired nitrogen atom substituent. In some embodiments, the alkyl halide or other compound with a good leaving group provides a nitrogen substituent which can increase the solubility of the heteroatom-doped acene in polar solvents (e.g., polar aprotic solvents, such as acetonitrile). Reaction of OFPT with MEEBr in the presence of a strong base, such as NaH, for instance, provides N-methyethoxyethyl octafluorophenothiazine (MEEOFPT).

Continuing the synthesis as shown in Scheme 2, below, the N-substituted OFPT can then undergo an aromatic nucleophilic substitution reaction (SNAr) (e.g., on both sides of the OFPT core) with a suitable reagent. Suitable reagents for the SNAr include aromatic and heteroaromatic compounds that comprise at least two nucleophilic substituents, —ZH, e.g., —OH, —NH₂, and/or —SH, substituted on adjacent carbon atoms. In some embodiments, the substituted aromatic or heteroaromatic compound can comprise a substituted benzene or fused aromatic compound comprising at least one fused benzene ring, wherein said substituted aromatic compound further comprises at least two nucleophilic substituents selected from —OH, —NH₂, and —SH, wherein the at least two nucleophilic substituents are substituted on neighboring (i.e., adjacent) carbon atoms of the same benzene or fused benzene ring. Suitable fused benzene ring scaffolds for the substituted aromatic or heteroaromatic compound include benzofuran, benzothiophene, indole, quinoline, isoquinoline, carbazole, dibenzofuran, anthracene, and naphthalene. In addition to the at least two nucleophilic substituents, the substituted aromatic compound can further include one or more additional aryl group substituents, e.g., alkyl, alkoxy, aralkyl, aryl, etc. In some embodiments, the reagent for use in the SNAr reaction comprises a substituted benzene, a substituted quinoline (e.g., substituted quinoline or substituted isoquinoline), a substituted benzofuran, a substituted benzothiophene, or a substitute benzopyrrole, wherein the substituents include at least two —ZH groups (i.e., —O—, —SH, or —NH) on adjacent benzene ring carbon atoms. In some embodiments, the SNAr reaction can be performed using about 2 molar equivalents of the reagent with the adjacent —ZH groups. In some embodiments, the SNAr reaction can be performed in a polar aprotic solvent. In some embodiments, the SNAr reaction can be performed in the presence of a base, such as potassium carbonate.

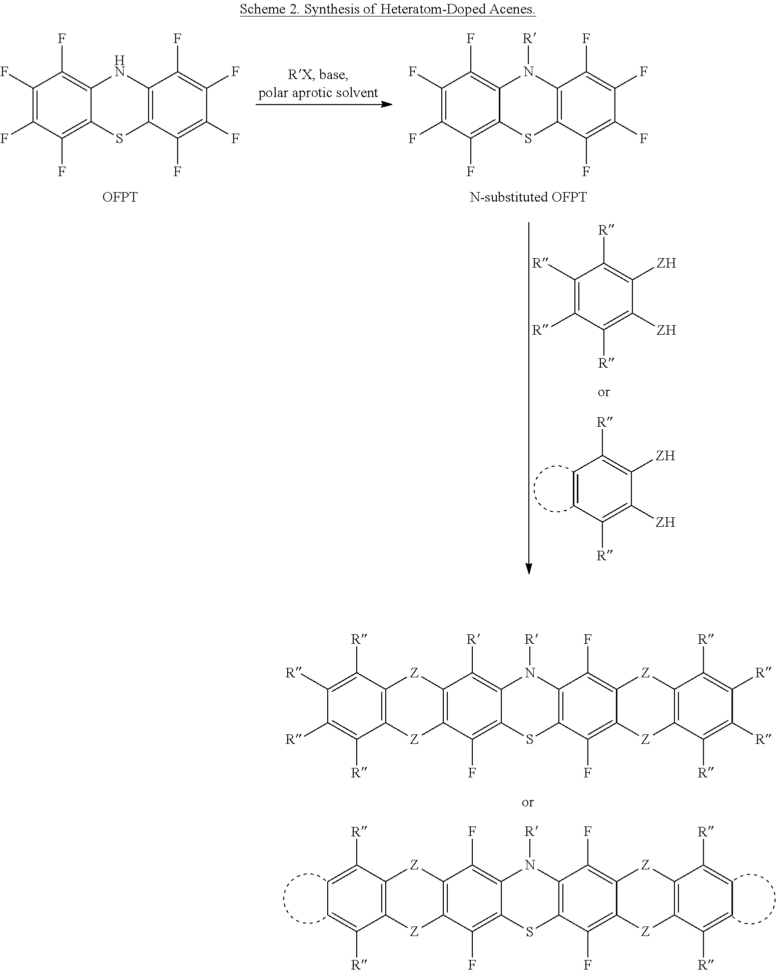

Accordingly, in some embodiments, the presently disclosed subject matter provides a method of preparing a heteroatom-doped acene, wherein the method comprises: (a) contacting octafluorophenothiazine (OFPT) with a first reagent in the presence of a hydride base, wherein said first reagent has a structure selected from:

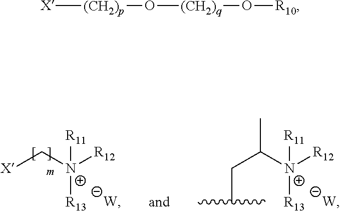

wherein X′ is selected from the group including Cl, Br, I, toluenesulfonate (Ots), or methanesulfonate (Oms); p, q, and m are each integers, e.g., independently selected from 1, 2, and 3; and R₁₀, R₁₁, R₁₂, and R₁₃ are each independently selected from H, alkyl, and benzyl; thereby providing a N-substituted OFPT; and (b) performing an aromatic nucleophilic substitution reaction by contacting the N-substituted OFPT with a substituted aromatic compound, wherein the substituted aromatic compound comprises a substituted benzene or fused aromatic compound comprising at least one fused benzene ring, and wherein said substituted aromatic compound further comprises at least two nucleophilic substituents selected from —OH, —NH₂, and —SH, wherein said at least two nucleophilic substituents are substituted on neighboring carbon atoms of the same benzene or fused benzene ring, thereby providing a heteroatom-doped acene, wherein said heteroatom-doped acene comprises a fused ring moiety comprising at least seven fused rings, wherein said at least seven fused rings together comprise least six heteroatorns selected from O, N, S, and B, and wherein the at least six heteroatoms comprise at least one N atom and at least one S atom.

In some embodiments, p and q are each 2. In some embodiments, R₁₀ is C1-C5 alkyl. In some embodiments, R₁₀ is methyl. In some embodiments, the first reagent is MEEBr. In some embodiments, the hydride base is sodium hydride (NaH).

In some embodiments, the substituted aromatic compound is a substituted benzene. In some embodiments, the substituted aromatic compound is selected from benzene-1,2-dithiol and benzene-1,2-diol.

In some embodiments, the heteroatom-doped acene is selected from:

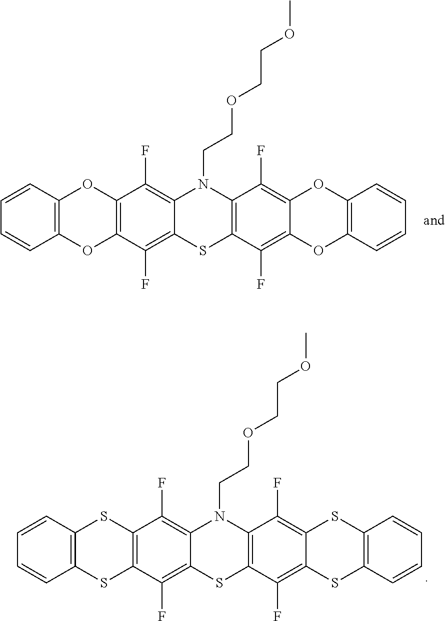 [SMILES: COCCOCCn1c2c(F)c3oc4ccccc4oc3c(F)c2sc2c(F)c3oc4ccccc4oc3c(F)c21 ; COCCOCCn1c2c(F)c3sc4ccccc4sc3c(F)c2sc2c(F)c3sc4ccccc4sc3c(F)c21]

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Synthesis of N,S-Doped Ace

The synthesis of N,S-doped heptacene was carried out starting from octafluorophenothiazine (OFPT), which was synthesized as previously described in the literature. See Kaur et al., 2016. See also Scheme 3, below. Aromatic nucleophilic substitution (SNAr) of OFPT with 1,2-benzenedithiol was attempted using sodium hydride (NaH) as base in dimethylformamide (DMF) for up to 72 hours. No conversion of starting material was observed under this condition. See Scheme 4, below, top. Synthesis of the desired acenes was then conducted by first substituting the amine functional group in OFPT with methoxyethoxyethyl bromide (MEEBr) to get N-methyethoxytheyl octafluorophenothiazine (MEEOFPT). See Kaur et al, 2016 and Schemes 3 and 4, below.

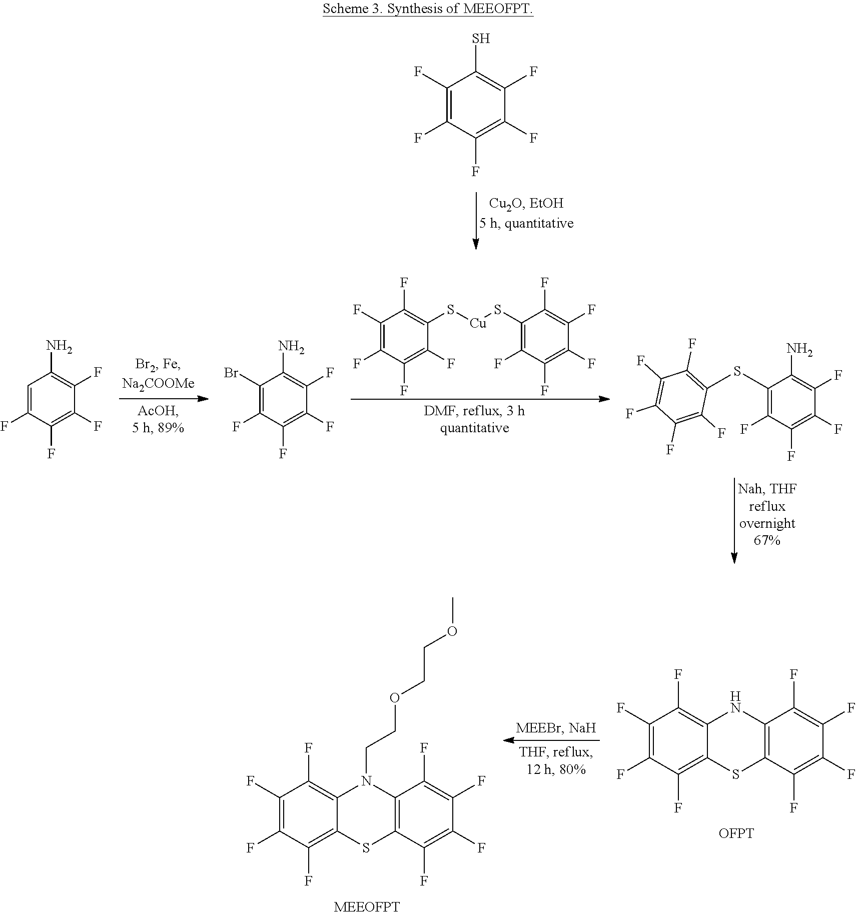

The N-substituted MEEOFPT was taken through an SNAr reaction under similar conditions to the prior attempt with OFPT. SNAr of the N-substituted MEEOFPT and 1,2-benzenedithiol afforded MEETFDTBPT in 80% yield as a light brown solid. SNAr of the N-substituted MEEOFPT and 1,2-benzenediol afforded the tetraoxygen version of the MEETFDTBPT, i.e., MEETFDOBPT. See Scheme 4, below. Additional synthetic details are described below.

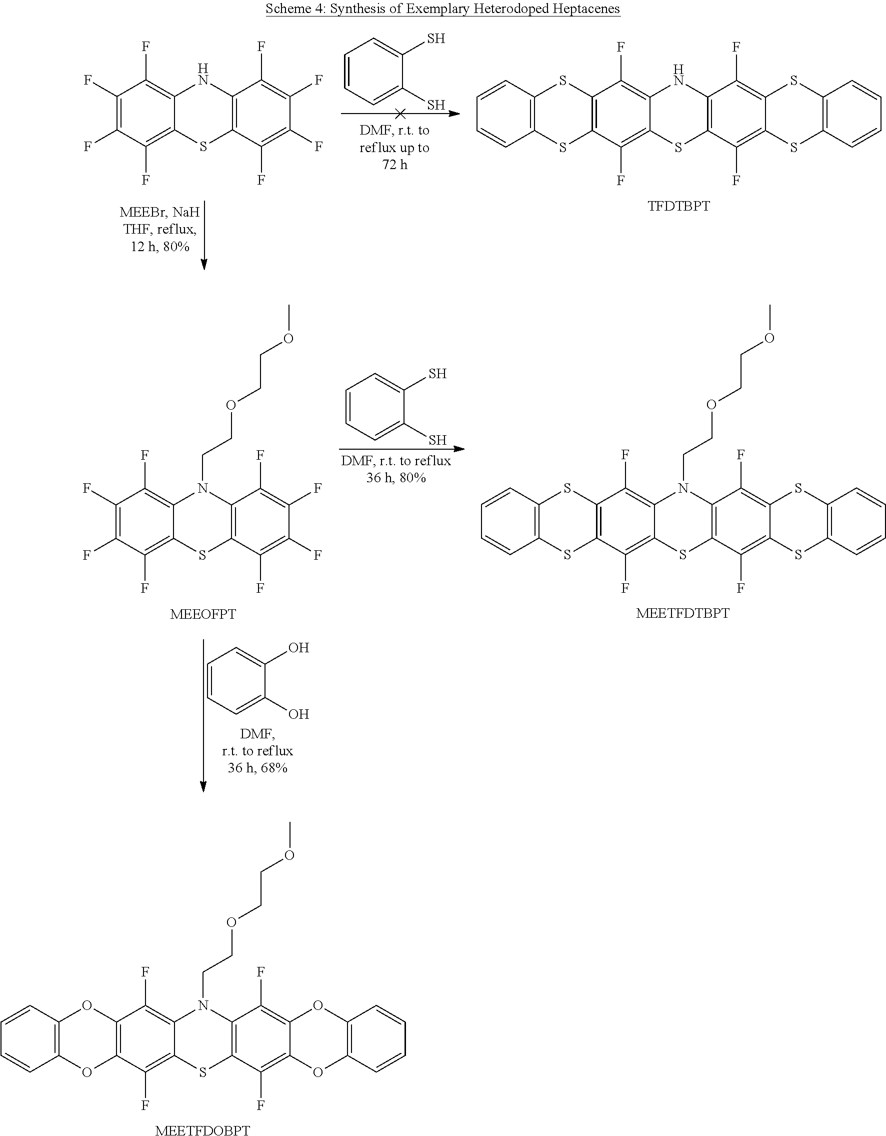

### Product Characterization

The products of the SNAr reactions were characterized with spectroscopic techniques such as NMR and IR spectroscopy and elemental analysis. There are three possible products that could be formed by the reaction of MEEOFPT with benzene-1,2-dithiol. The SNAr type reaction described above occurred regioselectively, forming the linear product among the three possible regioisomers, as confirmed by NMR analysis.

### Synthesis of MEETFDTBPT

In an oven-dried round bottom flask equipped with a magnetic stirrer, MEEOFPT (200 mg, 0.45 mmol, 1 equiv.) was dissolved in anhydrous DMF (10 mL). To this solution K₂CO₃ (186.20 mg, 1.35 mmol, 3 equiv.) and benzene-1,2-dithiol (127.75 mg, 0.89 mmol, 2 equiv.) were added under a nitrogen inert atmosphere. The reaction was stirred at room temperature for 24 h, after which solid product was precipitated by adding 1 M HCl. The crude product was filtered and washed with copius amount of water and methanol to yield MEETFDTBPT (250 mg, 80% yield) as a product. ¹H NMR (400 MHz, CDCl₃) δ 7.53-7.48, m, 4H), 7.31-7.25, m, 4H), 3.98-3.95 (m, 2H), 3.59-3.56 (m, 2H), 3.46-3.43 (m, 2H), 3.32-3.29 (m, 2H), 3.15 (s, 3H) ppm. ¹⁹F NMR (400 MHz, CDCl₃) δ 114.33, 114.38, 122.01, 122.06 ppm. ¹³C NMR (500 MHz, CDCl₃) δ 151.04 (m), 149.11 (m), 147.14 (m), 133.96, 132.39 (m), 129.37 (m), 128.43 (m), 124.62 (m), 119.58 (m), 117.84 (m), 71.86, 70.67, 69.65, 58.93, 53.94 ppm (m).

### Synthesis of MEETFDOBPT

In an oven-dried round bottom flask equipped with a magnetic stirrer, MEEOFPT (200 mg, 0.45 mmol, 1 equiv.) was dissolved in anhydrous DMF (10 mL). To this solution, K₂CO₃ (200 mg, 1.34 mmol, 3 equiv.) and benzene-1,2-diol (99 mg, 0.89 mmol, 2 equiv.) were added under an inert atmosphere (nitrogen). The reaction was stirred at room temperature for 24 h, after which solid product was precipitated by adding 1 M HCl. The crude product was filtered and washed with copius amount of water and methanol to yield MEETFDOBPT (187.08 mg, 71% yield) as a product. ¹H NMR (400 MHz, CDCl₃) δ 7.51-7.46, m, 4H), 7.30-7.22, m, 4H), 3.88-3.85 (m, 2H), 3.49-3.46 (m, 2H), 3.41-3.39 (m, 2H), 3.31-3.29 (m, 2H), 3.15 (s, 3H) ppm. ¹⁹F NMR (400 MHz, CDCl₃) δ 114.31, 114.36, 122.00, 122.02 ppm. ¹³C NMR (500 MHz, CDCl₃) δ 151.06 (m), 149.12 (m), 147.04 (m), 133.63, 132.31 (m), 129.23 (m), 128,14 (m), 124.42 (m), 119.38 (m), 117.34 (m), 71.85, 70.67, 69.66, 58.83, 53.91 ppm (m).

### Example 2

### Electrochemical Characterization

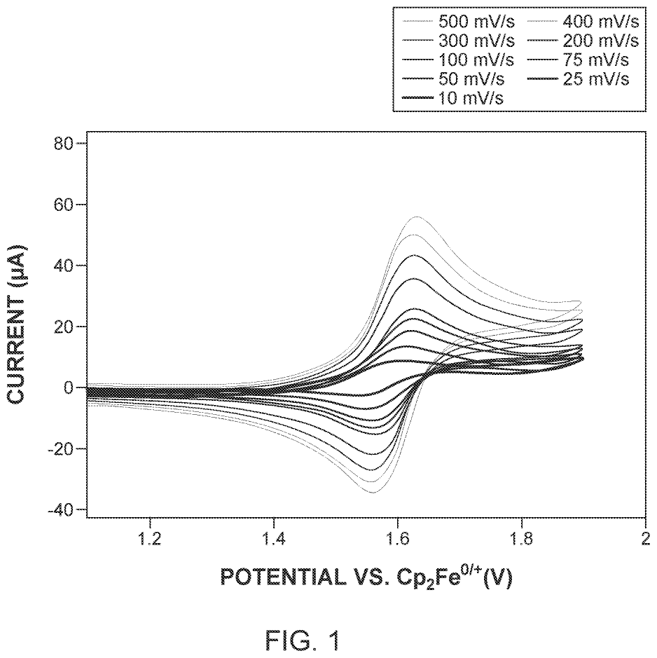

The oxidation potential of the heteroatom-doped heptacenes was determined using cyclic voltammetry. MEETFDOBPT did not exhibit reversible redox event. Accordingly, further characterization was not carried out for the tetraoxygen compound. For MEETFDTBPT, due to a solubility of the molecule of about 0.7 mM in acetonitrile, cyclic voltammetry was performed of a 1 mM solution in 0.1 M TEABF₄/acetonitrile. See FIG. 1. The oxidation potential of MEETFDTBPT was calculated to be 1.61 V referenced to Fc/Fc+, which is higher compared to common electrolytes reported in the literature for RFB or LIB applications. See Yan et al., 2022.

### Example 3

### Radical Cation Synthesis and Stability Study

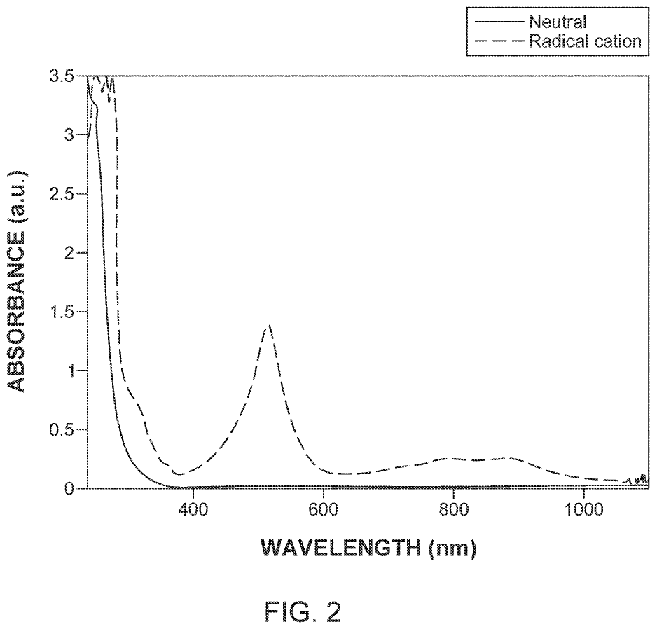

The radical cation of MEETFDTBPT was synthesized via chemical oxidation using silver bistriflimide (2.5 equiv.) as a chemical oxidant and anhydrous acetonitrile as solvent under an inert environment. See Scheme 5, below. The generation of radical cation was monitored by UV-vis spectroscopy as evidenced by the sudden appearance of an intense absorption peak in the 550 nm wavelength region. See FIG. 2. After stirring the reaction mixture for 10 min, the reaction was quenched with anhydrous ether, providing a deep-red viscous semisolid.

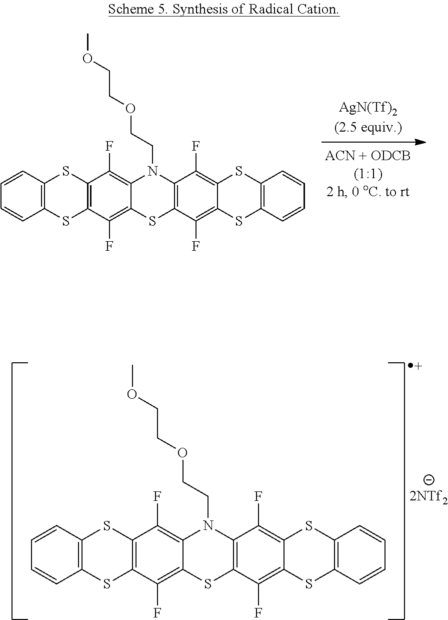

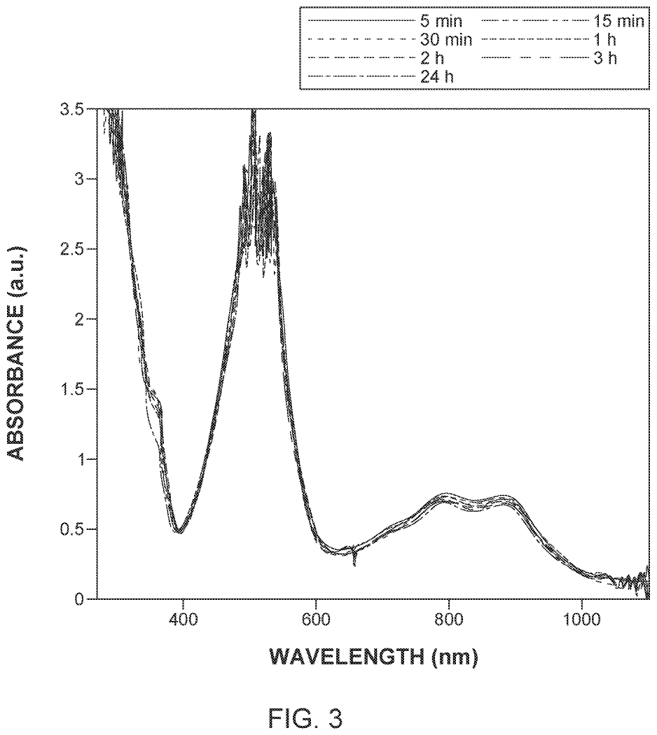

A 10 mM solution of the radical cation was tested for stability in anhydrous acetonitrile over 24 hours. See FIG. 3. There was no significant loss of absorption intensity measuring at different time such as 5 min, 15 min, 30 min, 1 h, 2 h, 3 h and 24 h.

### Example 4

### Solubility

The solubilities of the neutral and charged forms of the redox-active species were measured in anhydrous acetonitrile and 0.1 M TEABF₄ in acetonitrile. In case of the liquid and semi solid chemical species, miscibility in acetonitrile and electrolyte solution (i.e. 0.1 M TEABF₄ in acetonitrile) were measured. See Table 1, below.

TABLE 1
Solubility of Redoxamers

| | Solubility in | Solubility in |
|---|---|---|
| | acetonitrile | 0.1M TEABF₄ in |
| Redoxmer | (M) | acetonitrile (M) |
| MEETFDTBPT | 0.7 | 0.5 |
| MEETFDOBPT | 0.6 | 0.3 |
| MEETFDTBPT-2TFSI | miscible | miscible |

### REFERENCES

All references listed in the instant disclosure, including but not limited to all patents, patent applications and publications thereof, scientific journal articles, and database entries, are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Kaur, A. P.; Casselman, M. D.; Elliott, C. F.; Parkin, S. R.; Risko, C.; Odom, S. A. “Overcharge Protection of Lithium-Ion Batteries above 4V with a Perfluorinated Phenothiazine Derivative,” J. Mater. Chem. A, 2016, 4, 5410-5414.
Yan, Y.; Sitaula, P.; Odorn, S. A.; Vaid, T. P. “High Energy Density, Asymmetric, Nonaqueous Redox Flow Batteries without a Supporting Electrolyte,” ACS App. Mater. Interfaces, 2022, 14, 44, 49633-49640.

It will be understood that various details of the presently disclosed subject matter can be changed without departing from the scope of the presently disclosed subject matter. Furthermore, the foregoing description is for the purpose of illustration only, and not for the purpose of limitation.

1. A heteroatom-doped acene, wherein the heteroatom-doped acene comprises a fused ring moiety comprising at least seven fused rings, wherein said at least seven fused rings together comprise at least six heteroatoms selected from O, N, S, and B, and wherein the at least six heteroatoms comprise at least one N atom and at least one S atom.

2. The heteroatom-doped acene of claim 1, wherein the fused ring moiety comprises a central phenothiazine moiety.

3. The heteroatom-doped acene of claim 1, wherein the heteroatom-doped acene is a heteroatom-doped heptacene or longer acene.

4. The heteroatom-doped acene of claim 3, wherein the heteroatom-doped acene is a heteroatom-doped heptacene or longer acene doped with at least one N atom and at least five S atoms.

5. A heteroatom-doped acene, wherein the heteroatom-doped acene has a structure of Formula (I): 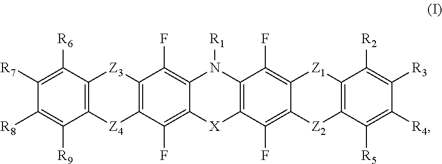
wherein:
X is selected from N, S, and B;
each of Z₁, Z₂, Z₃, and Z₄ is independently selected from O, N, and S;
R₁ is selected from —(CH₂)ₚ—O—(CH₂)_{q}—O—R₁₀, 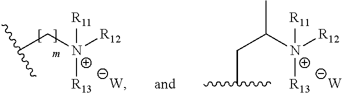
wherein p, q, and m are each an integer, independently selected from 1, 2, and 3; and R₁₀, R₁₁, R₁₂, and R₁₃ are independently selected from H, alkyl, and benzyl;
R₂-R₉ are each independently selected from H, alkyl, alkoxy, alkylamino, alkylthio, aralkyl, aryl, aryloxy, arylamino, and arylthiol,
or wherein R₇ and R₈ together with the carbon atoms to which they are attached and/or R₃ and R₄ together with the carbon atoms to which they are attached form a fused five- or six-membered aromatic or heteroaromatic ring, optionally selected from the group consisting of benzene, substituted benzene, pyridine, substituted pyridine, thiophene, furan, and pyrrole; and
W is an anionic moiety, optionally selected from the group consisting of bis(trifluoromethane)sulfonimide, (TFSI), tetrafluoroborate (BF₄), Br, Cl, I, trifluoromethanesulfonate (OTf), toluenesulfonate (OTs), and methanesulfonate (OMs).

6. The heteroatom-doped acene of claim 5, wherein at least one of X, Z₁, Z₂, Z₃, and Z₄ X is S.

7. The heteroatom-doped acene of claim 5, wherein X is S.

8. The heteroatom-doped acene of claim 5, wherein Z₁, Z₂, Z₃, and Z₄ are each S.

9. The heteroatom-doped acene of claim 5, wherein X is S and each of Z₁, Z₂, Z₃, and Z₄ is S and the heteroatom-doped acene of Formula (I) has a structure of Formula (II): 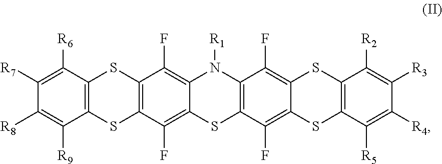
wherein R₁-R₉ are as defined for Formula (I).

10. The heteroatom-doped acene of claim 5, wherein R₁ is —(CH₂)ₚ—O—(CH₂)_{q}—O—R₁₀, wherein p and q are each 2.

11. (canceled)

12. The heteroatom-doped acene of claim 5, wherein R₂-R₉ are each independently selected from H, C1-C5 alkyl, and C1-C5 alkoxy.

13. (canceled)

14. The heteroatom-doped acene of claim 5, wherein R₇ and R₈ together with the carbon atoms to which they are attached and R₃ and R₄ together with the carbon atoms to which they are attached each form a fused five- or six-membered aromatic or heteroaromatic ring, optionally selected from the group consisting of benzene, substituted benzene, pyridine, substituted pyridine, thiophene, furan, and pyrrole.

15. The heteroatom-doped acene of claim 5, wherein the heteroatom-doped acene has a structure selected from:

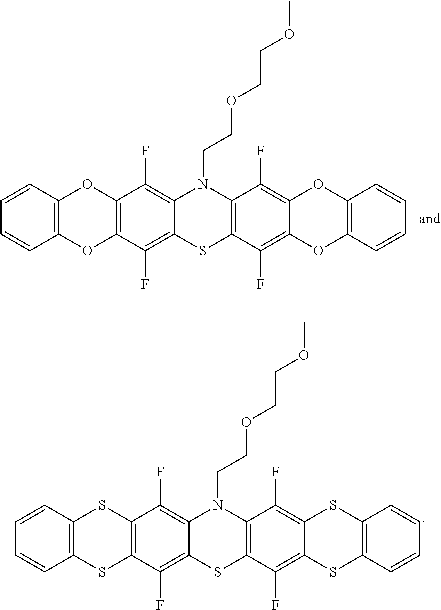 [SMILES: COCCOCCn1c2c(F)c3oc4ccccc4oc3c(F)c2sc2c(F)c3oc4ccccc4oc3c(F)c21 ; COCCOCCn1c2c(F)c3sc4ccccc4sc3c(F)c2sc2c(F)c3sc4ccccc4sc3c(F)c21]

16. The heteroatom-doped acene of claim 5, wherein the heteroatom-doped acene can undergo a reversible redox reaction.

17. The heteroatom-doped acene of claim 5, wherein the heteroatom-doped acene is chemically stable in both charged and neutral forms.

18. The heteroatom-doped acene of claim 5, wherein the heteroatom-doped acene has an oxidation potential of about 0.7 V or more referenced to ferrocene (Fc)/ferrocenium (Fc+), optionally wherein the heteroatom-doped acene has an oxidation potential of about 0.7 V to about 1.75 V referenced to Fc/Fc+.

19-22. (canceled)

23. A method of preparing a heteroatom-doped acene, wherein the method comprises:
(a) contacting octafluorophenothiazine (OFPT) with a first reagent in the presence of a hydride base, wherein said first reagent has a structure selected from: 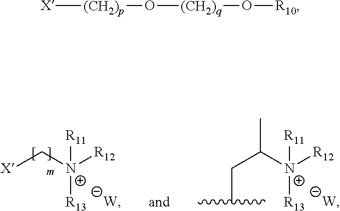
wherein X′ is Cl, Br, I, toluenesulfonate (OTs), or methanesulfonate (OMs); p, q, and m are each integers, independently selected from 1, 2, and 3; and R₁₀, R₁₁, R₁₂, and R₁₃ are each independently selected from H, alkyl, and benzyl; thereby providing an N-substituted OFPT; and
(b) performing an aromatic nucleophilic substitution reaction by contacting the N-substituted OFPT with a substituted aromatic compound, wherein the substituted aromatic compound comprises a substituted benzene or fused aromatic compound comprising at least one fused benzene ring, and wherein said substituted aromatic compound further comprises at least two nucleophilic substituents selected from —OH, —NH₂, and —SH, wherein said at least two nucleophilic substituents are substituted on neighboring carbon atoms of the same benzene or fused benzene ring, thereby providing a heteroatom-doped acene, wherein said heteroatom-doped acene comprises a fused ring moiety comprising at least seven fused rings, wherein said at least seven fused rings together comprise at least six heteroatoms selected from O, N, S, and B, and wherein the at least six heteroatoms comprise at least one N atom and at least one S atom.

24. The method of claim 23, wherein the first reagent is methoxyethoxyethyl bromide (MEEBr).

25. The method of claim 23, wherein the hydride base is sodium hydride (NaH).

26. The method of claim 23, wherein the substituted aromatic compound is selected from benzene-1,2-dithiol and benzene-1,2-diol.

27. (canceled)
